(19) United States
(12) Patent Application Publication
(10) Pub. No.: US 2002/0111473 A1
(43) Pub. Date: Aug. 15, 2002

(54) NOVEL G PROTEIN COUPLED RECEPTOR

(76) Inventors: ROMAN L. ZASTAWNY, ETOBICOKE (CA); ELIZABETH A. MCWHINNIE, ETOBICOKE (CA)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 09/292,973

(22) Filed: Apr. 16, 1999

Related U.S. Application Data
(60) Provisional application No. 60/081,995, filed on Apr. 16, 1998.

(51) Int. Cl. G01N 33/53; C07H 21/04; C12P 21/06; C12N 5/00; C12N 5/02

(52) U.S. Cl. 536/23.5; 435/325; 435/7.1; 536/24.31

(57) ABSTRACT

A novel G protein coupled receptor family is described, herein called B5. DNA coding for members of this family has been isolated. Methods of producing recombinant cell lines which produce the receptors as a heterologous membrane-bound product are described, as well as other related aspects of the invention, which are of commercial significance, including use of the cell lines as a tool for the discovery of therapeutic compounds which modulate the receptor activity.

### FIELD OF THE INVENTION

This invention is concerned with applications of recombinant DNA technology. More particularly, the invention relates to the cloning and expression of DNA coding for novel G protein coupled receptors.

### BACKGROUND TO THE INVENTION

G protein coupled receptors have been implicated in many important biological processes in a wide variety of living organisms and include a wide range of biologically active receptors, such as hormone, growth factor and neuroreceptors. For example, adrenergic agents and dopamine (Kobilka et al, PNAS, 84:46-50 (1987); Kobilka et al. Science, 238:650-656 (1987); Bunzow et al, Nature 336:783-787 (1998)); calcitonin; cAMP; adenosine; muscarinic; serotonin all act through G protein coupled receptors.

Members of this class share a common signalling mechanism which involves intracellular transducer elements called G proteins. Briefly, when a chemical messenger binds to the active site of the receptor, the conformation of the receptor changes thereby allowing it to interact with and activate a G protein. The activated G protein causes a molecule of guanosine diphosphate (GDP), that is bound to the surface of the G protein, to be replaced with a molecule of guanosine triphosphate, which causes another alteration in the conformation of the G protein. With GTP bound to its surface the G protein can regulate the activity of an effector. These effectors include enzymes such as adenylyl cyclase and phospholipase C, certain transport proteins and ion channels such as those specific for calcium ions, potassium ions or sodium ions.

G protein coupled receptors have been characterised as having seven putative transmembrane domains each of the order of 20 to 30 hydrophobic amino acids, connecting at least eight divergent hydrophilic loops. The transmembrane regions are designated TM1, TM2 etc. TM3 is implicated in ligand binding signal transduction. Additionally, TM5 and TM6 are implicated in ligand binding. Post translational events such as phosphorylation and lipidation can influence receptor activity.

In view of the diverse functions of G protein coupled receptors, it is not surprising many therapeutic drugs act by directly modifying the function of G protein coupled receptors.

### SUMMARY OF THE INVENTION

The present invention relates to an isolated polynucleotide sequence encoding a novel mammalian G protein coupled receptor. In one of its aspects the invention thus provides an isolated polynucleotide, consisting either of DNA or of RNA, which codes for a G protein coupled receptor or for a fragment or variant thereof.

In another aspect of the present invention, there is provided a cell that has been genetically engineered to produce a G protein coupled receptor herein defined as a member of the B5 family. In related aspects of the present invention, there are provided recombinant DNA constructs and relevant methods useful to create such cells.

In another aspect of the present invention, there is provided a method for evaluating interaction between a test ligand and a B5 receptor, which comprises the steps of incubating the test ligand with a cell engineered genetically to produce a B5 receptor, or with a membrane preparation derived therefrom, and then assessing said interaction by determining at least one of receptor/ligand binding, ligand-induced current, or second messenger response, such as modulation of cAMP or intracellular calcium levels.

Other aspects of the present invention, which encompass various applications of the discoveries herein described, will become apparent from the following detailed description, and from the accompanying drawings, in which:

### BRIEF REFERENCE TO THE FIGURES

FIG. 1 provides a nucleotide acid sequence encoding the rat B5 receptor and the predicted amino acid sequence

FIG. 2 provides a nucleotide sequence of DNA encoding the partial human B5 receptor and the amino acid sequence

FIG. 3 shows the percentage similarity and identity between the amino acid sequence of the rat B5 receptor and closely related G protein-coupled receptors

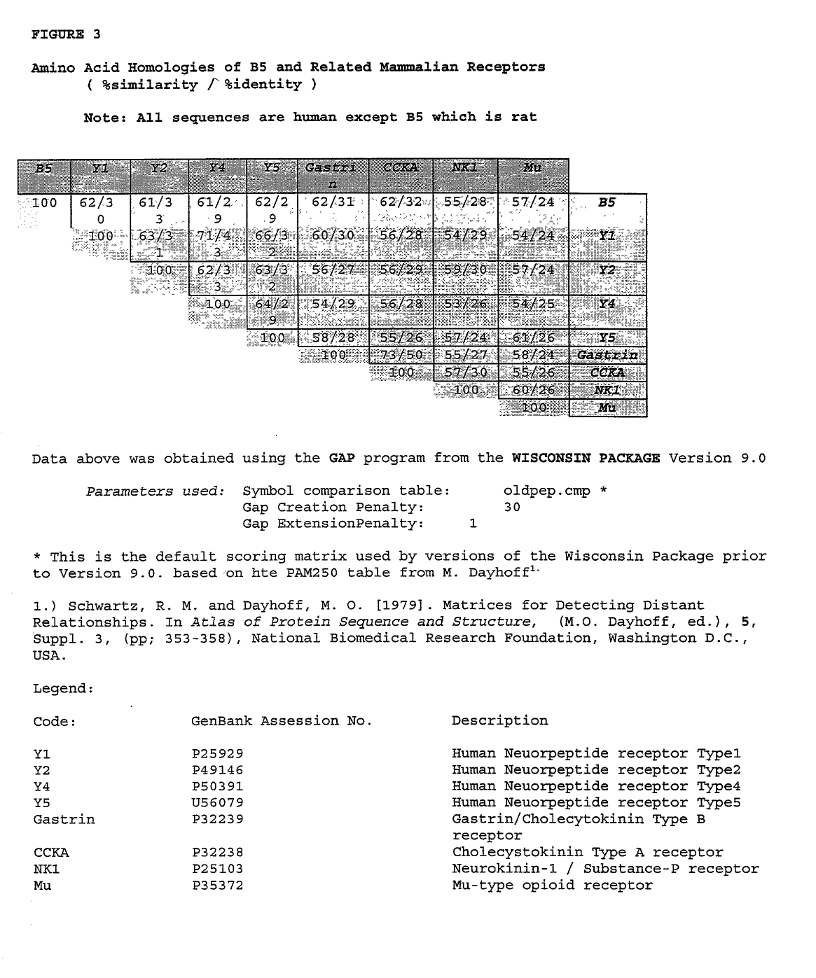

FIG. 4 shows a comparison between the predicted amino acid sequence of the rat B5 receptor of FIG. 1 and the partial amino acid sequence of the human B5 receptor of FIG. 2.

FIG. 5 shows a comparison between the amino acid sequence of the rat B5 receptor and the human Y2 receptor.

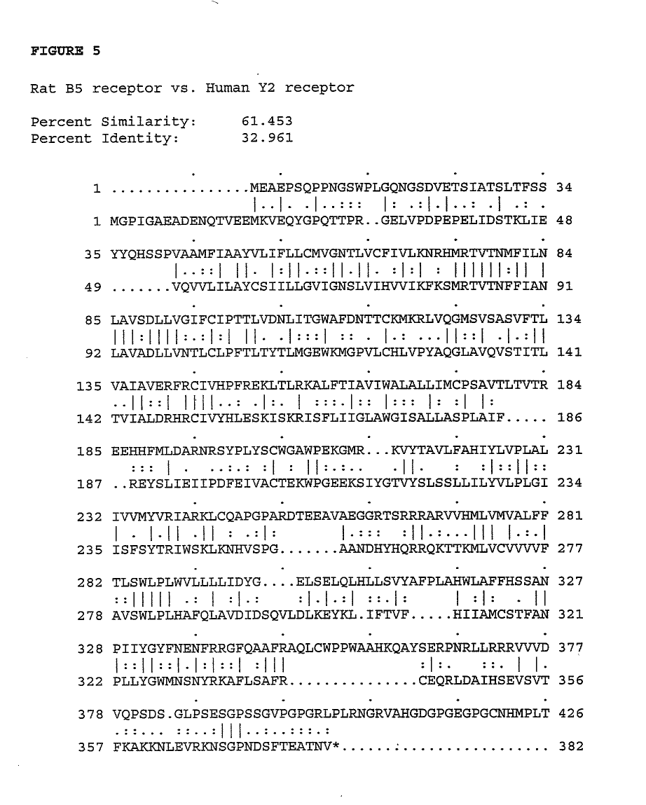

FIG. 6 illustrates the FISH mapping results for the B5 receptor/probe 248 on human chromosome 10.

### DETAILED DESCRIPTION OF THE INVENTION AND ITS PREFERRED EMBODIMENTS

The invention relates to G-protein coupled receptors of mammalian origin, including human, and is directed more particularly to a novel G protein coupled receptor, herein designated the B5 receptor, and to isolated polynucleotides encoding these receptors. As used herein “isolated” means separated from polynucleotides that encode other proteins. In the context of polynucleotide libraries, for instance, a B5 receptor-encoding polynucleotide is considered “isolated” when it, or a clone incorporating it, has been selected, and hence removed from association with other polynucleotides within the library. Such polynucleotides may be in the form of RNA, or in the form of DNA including cDNA, genomic DNA and synthetic DNA.

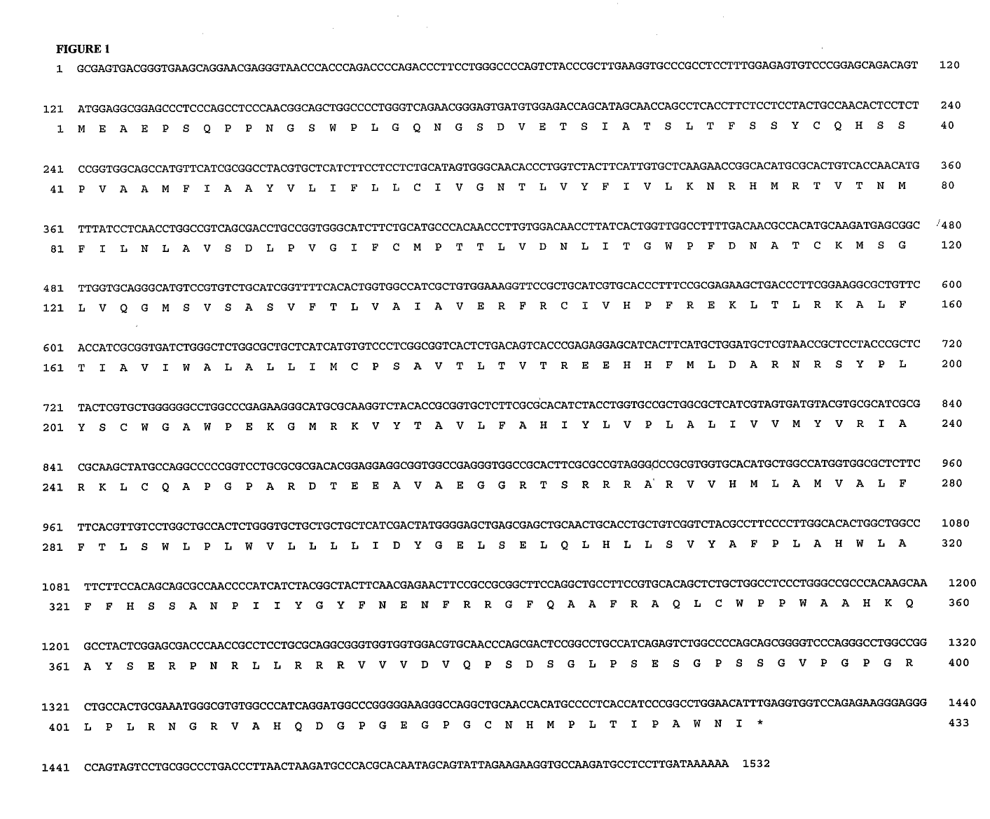

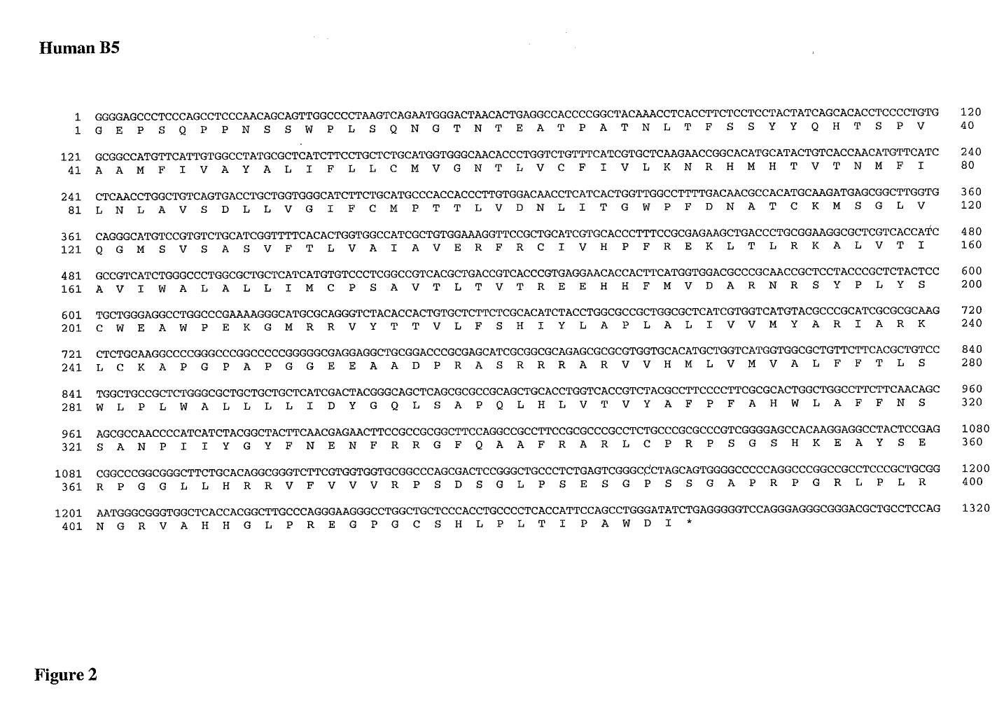

The present invention further relates to variants of the B5 polynucleotides described hereinwhich encode fragments, analogs and derivatives of the peptides having the derived amino acid sequence of FIG. 1 or FIG. 2. The variants of the polynucleotides may be naturally occurring allelic variants or non-naturally occurring variants of the polynucleotides wherein the synonymous codon is substituted for the native sequence.

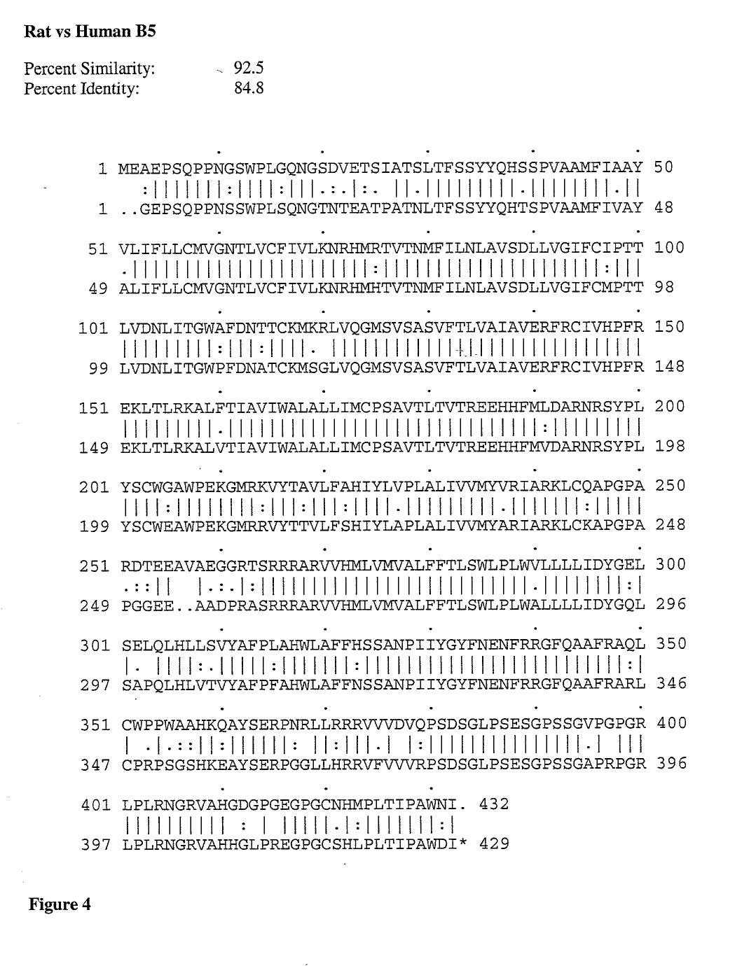

As used herein, the term “B5 receptor” is intended to embrace rat receptors and functional variants that are structurally related thereto, i.e. share at least 34% amino acid identity therewith, including naturally occurring and synthetically derived variants. Naturally occurring variants include mammalian species homologues of the rat B5 receptor and in particular include the human B5 receptor. Synthetically derived variants of the B5 receptor include ligand binding variants that incorporate one or more, e.g. 1-10, amino acid substitutions, deletions or additions, relative to the rat or human or naturally occurring variants of the rat receptor. Generally, it will be desirable that such synthetic variants retain the ligand binding and signal transducing activities of the naturally occurring receptor. Therefore, preferably above-mentioned substitutions, deletions or additions will be conservative in nature i.e. relate to positions in the amino acid sequence wherein such modifications do not result in complete loss of receptor function, that is ligand binding and/or ability to signal transduce. Alignment of the rat and human B5 amino acid sequences provided herein (FIG. 4) indicates points in these sequences where it is expected that modifications may be made without loss of function.

As used herein the terms fragment, derivative and analog means a polypeptide which either retains substantially the same biological function or activity of B5 i.e functions as a G protein coupled receptor, or retains the ability to bind the ligand, for example a soluble form of the receptor. Fragments also include portions of the B5 protein which are useful for raising antibodies, detailed hereinbelow.

Like other members of the G protein coupled receptor family, receptor subtype B5 is characterised by a pharmacological profile i.e. a ligand binding “signature”. Thus, in a key aspect of the present invention, the B5 receptor is exploited for the purpose of screening candidate ligands, including candidate drug compounds, which have the ability to interact with the present receptors and/or the ability to compete with endogenous B5 receptor ligands. In one embodiment preferably, candidate ligands to be screened are peptides. In a more preferred embodiment candidate ligands are NPY, peptide YY, CCK, gastrin, substance P or substance K. Most preferably, candidate ligands are NPY, CCK or gastrin and pepetide analogs of those.

A polynucleotide encoding a polypeptide of the present invention has been found in rat brain, testis, skeletal muscle, colon, pancreas and adipose tissue. The human polynucleotide of the invention were isolated from a BAC human genomic library. The rat polynucleotide was isolated from a cDNA library of hypothalamal origin. It is structurally related to the G protein coupled receptor family. It contains an open reading frame encoding a protein of 432 amino acids. The B5 receptor protein exhibits the highest degree of homology to the NPY Y2 receptor family with 33% identity and 61% similarity over the entire amino acide sequences to the NPY Y2 receptor. B5 also shows significant homology to the Gastrin and CCKA receptors as well as an orphan receptor (WO 9634877). These receptors possess structural features characteristic of the G protein coupled receptors in general, including an extracellular N-terminus and an intracellular C-terminus, as well as seven transmembrane domains which serve to anchor the receptor within the cell surface membrane. These receptors are further characterised by their coupling to G-proteins, or guanine nucleotide regulatory proteins. With respect to structural domains of the rat B5 receptor, hydropathy analysis reveals seven putative transmembrane domains, one spanning residues 46-69 inclusive (TM-1), another spanning residues 80-102 (TM-2), a third spanning residues 118-139(TM-3), a fourth spanning residues 158-179 (TM-4), a fifth spanning residues 215-237(TM-5), a sixth spanning residues 273-295 (TM-6) and a seventh spanning residues 311-334 (TM-7). Based on this assignment, it is likely that the B5 receptor structure, in its natural membrane-bound form, consists of a 45 amino acid N-terminal extracellular domain, followed by a hydrophobic region containing seven transmembrane domains and an intracellular 98 amino acid C-terminal domain.

The invention also relates to polynucleotides which hybridise to the hereinabove described sequences if there is at least 46% and preferably 55% homology between B5 and the hybridising sequences. More preferably the hybridising sequences show at least 70% homology to the sequences described herein and most preferably at least 84% homology. In particular, the invention relates to polynucleotides which hybridise under conditions of high stringency to the described B5 polynucleotides. As used herein conditions of high stringency means hybridisation will occur only if there is at least 84%, preferably 90% and more preferably 95% identity between the sequences. In a preferred embodiment, the polynucleotides which hybridise to the B5 encoding polynucleotides either retain substantially the same biological function or activity as B5 i.e function a G protein coupled receptor, or retain the ability to bind the ligand for the receptor even though the polypeptide does not function as a G protein coupled receptor, for example the soluble for of the receptor.

“Identity” or “Sequence identity” is known in the art, and is a relationship between two or more polypeptide sequences or two or more polynucleotide sequences, as determined by comparing the sequences, particularly, as determined by the match between strings of such sequences. Sequence identity can be readily calculated by known methods (Computational Molecular Biology, Lesk, A. M., ed., Oxford University Press, New York, 1988; Biocomputing: informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exist a number of methods to measure identity between two sequences, the term is well known to skilled artisans (see, for example, Sequence Analysis in Molecular Biology; Sequence Analysis Primer; and Carillo, H., and Lipman, D., SIAM J. Applied Math., 48: 1073 (1988)). Methods commonly employed to determine identity between sequences include, but are not limited to those disclosed in Carillo, H., and Lipman, D., SIAM J. Applied Math., 48: 1073 (1988) or, in Needleman and Wunsch, J. Mol. Biol., 48: 443-445, 1970, wherein the parameters are as set in version 2 of DNASIS (Hitachi Software Engineering Co., San Bruno, Calif.). Computer programs for determining identity are publicly available. Preferred computer program methods to determine identity between two sequences include, but are not limited to, GCG program package (Devereux, J., et al., Nucleic Acids Research 12(1): 387 (1984)), BLASTP, BLASTN, and FASTA (Atschul, S. F. et al., J. Molec. Biol. 215: 403-410 (1990)) or using the GAP program from the WISCONSIN PACKAGE Version 9.0. The BLASTX program is publicly available from NCBI (blast@ncbi.nhn.nih.gov) and other sources (BLAST Manual, Altschul, S., et al., NCBI NLM NIH Bethesda, Md. 20894; Altschul, S., et al., J. Mol. Bio. 215: 403-410 (1990)).Computational Molecular Biology, Lesk, A. M, ed. Unless specified otherwise in the claims, the percent identity for the purpose of interpreting the claims shall be calculated by or using the GAP program from the WISCONSIN PACKAGE Version 9 wherin the parameters used are as follows:

| Symbol comparison table: | oldpep.cmp* |
|---|---|
| Gap Creation Penalty: | 30 |
| Gap ExtensionPenalty: | 1 |

This is the default scoring matrix used by versions of the Wisconsin Package prior to Version 9.0. based on hte PAM250 table from Schwartz, R. M. and Dayhoff, M. O. [1979]. Matrices for Detecting Distant Relationships. In Atlas of Protein Sequence and Structure, (M. O. Dayhoff, ed.), 5, Suppl. 3, (pp; 353-358), National Biomedical Research Foundation, Washington D.C., USA.

For use in assessing interaction between the receptor and a candidate ligand, it is desirable to construct by application of genetic engineering techniques a mammalian cell that produces a B5 receptor in functional form as a heterologous product. The construction of such cell lines is achieved by introducing into a selected host cell a recombinant DNA construct in which DNA coding for the B5 receptor is associated with expression controlling elements that are functional in the selected host to drive expression of the receptor-encoding DNA, and thus elaborate the desired B5 receptor protein. Such cells are herein characterised as having the receptor-encoding DNA incorporated “expressibly” therein. The receptor-encoding DNA is referred to as “heterologous” with respect to the particular cellular host if such DNA is not naturally found in the particular host.

The particular cell type selected to serve as host for production of the B5 receptor can be any of several cell types currently available in the art, including both prokaryotic and eukaryotic, but desirably is not a cell type that in its natural state elaborates a surface receptor that binds B5 ligand, or analogues thereof, so as to confuse the assay results sought from the engineered cell line. Generally, such problems are avoided by selecting as host cell type which does not express significant levels of B5, for example, lung, kidney or ovary. Such problems can further be avoided by selecting a non-mammalian cell as a starting material for the analysis. However, it will be appreciated that mammalian cells may nevertheless serve as expression hosts, provided that “background” binding to the test ligand is accounted for in the assay results. In the alternative, the B5 sequence informaton herein disclosed allows for the identification of cells expressing endogenous B5 receptor, and hence alows for their selection and use in compound screening programs. The use of such B5 receptor producing cells in a screening program is also within the scope of the invention.

According to one embodiment of the present invention, the cell line selected to serve as host for B5 receptor production is a mammalian cell. Several types of such cell lines are currently available for genetic engineering work, and these include the Chinese hamster ovary (CHO) cells for example of K1 lineage (ATCC CCL 61) including the Pro5 variant (ATCC CRL 1281); the fibroblast-like cells derived from SV40-transformed African Green monkey kidney of the CV-1 lineage (ATCC CCL 70), of the COS-1 lineage (ATCC CRL 1650) and of the COS-7 lineage (ATCC CRL 1651); murine L-cells, murine 3T3 cells (ATCC CRL 1658), murine C127 cells, human embryonic kidney cells of the 293 lineage (ATCC CRL 1573), human carcinoma cells including those of the HeLa lineage (ATCC CCL 2), and neuroblastoma cells of the lines IMR-32 (ATCC CCL 127), SK-N-MC (ATCC HTB 10) and SK-N-SH (ATCC HTB 11).

A variety of gene expression systems have been adapted for use with these hosts and are now commercially available, and any one of these systems can be selected to drive expression of the B5 receptor-encoding DNA. These systems, available typically in the form of plasmidic vectors, incorporate expression cassettes the functional components of which include DNA constituting expression controlling sequences, which are host-recognized and enable expression of the receptor-encoding DNA when linked 5′ thereof. The systems further incorporate DNA sequences which terminate expression when linked 3′ of the receptor-encoding region. Thus, for expression in the selected mammalian cell host, there is generated a recombinant DNA expression construct in which DNA coding for the receptor is linked with expression controlling DNA sequences recognized by the host, and which include a region 5′ of the receptor-encoding DNA to drive expression, and a 3′ region to terminate expression. The plasmidic vector harbouring the expression construct typically incorporates such other functional components as an origin of replication, usually virally-derived, to permit replication of the plasmid in the expression host and desirably also for plasmid amplification in a bacterial host, such as E.coli. To provide a marker enabling selection of stably transformed recombinant cells, the vector will also incorporate a gene conferring some survival advantage on the transformants, such as a gene coding for G418 resistance in which case the transformants are plated in medium supplemented with G418.

Included among the various recombinant DNA expression systems that can be used to achieve mammalian cell expression of the receptor-encoding DNA are those that exploit promoters of viruses that infect mammalian cells, such as the promoter from the cytomegalovirus (CMV), the Rous sarcoma virus (RSV), simian virus (SV40), murine mammary tumour virus (MMTV) and others. Also useful to drive expression are promoters such as the LTR of retroviruses, insect cell promoters such as those regulated by temperature, and isolated from Drosophila, as well as mammalian gene promoters such as those regulated by heavy metals, i.e. the metallothionein gene promoter, and other steroid-inducible promoters.

For incorporation into the recombinant DNA expression vector, DNA coding for the desired B5 receptor, can be obtained by applying selected techniques of gene isolation or gene synthesis. The human B5 receptor is expressed in human brain tissue, and can therefore be obtained by careful application of conventional gene isolation and cloning techniques. This typically will entail extraction of total messenger RNA from a fresh source of human brain tissue, such as hypothalamus or hindbrain tissue followed by conversion of message to cDNA and formation of a library in for example a bacterial plasmid, more typically a bacteriophage. Such bacteriophage harbouring fragments of the human DNA are typically grown by plating on a lawn of susceptible E. coli bacteria, such that individual phage plaques or colonies can be isolated. The DNA carried by the phage colony is then typically immobilized on a nitrocellulose or nylon-based hybridisation membrane, and then hybridized, under carefully controlled conditions, to a radioactively (or otherwise) labelled oligonucleotide probe of appropriate sequence to identify the particular phage colony carrying receptor-encoding DNA or fragment thereof. Typically, the gene or a portion thereof so identified is subcloned into a plasmidic vector for nucleic acid sequence analysis.

An acceptable alternative to using the hybridisation screening method described above for isolating the desired B5 DNA is the PCR homology method. This method of PCR is described in detail in the examples herein. Generally this method involves the amplification of DNA containing specific sequences which are selected via hybridisation to specific primer sequences.

In a specific embodiment of the invention, the B5 receptor is encoded by the rat DNA sequence illustrated in FIG. 1 and the partial human DNA sequence illustrated in FIG. 2.

In obvious alternatives, the DNA sequences of FIG. 1 and FIG. 2 may be modified to incorporate synonymous codon equivalents while maintaining a DNA sequence that encodes the B5 receptor.

Having herein provided the partial nucleotide sequence of a human B5 receptor, it will be appreciated that automated techniques of gene synthesis and/or amplification can be performed to generate DNA coding therefor. Because of the length of B5 receptor-encoding DNA, application of automated synthesis may require staged gene construction, in which regions of the gene up to about 300 nucleotides in length are synthesized individually and then ligated in correct succession for final assembly. Individually synthesized gene regions can be amplified prior to assembly, using polymerase chain reaction (PCR) technology.

The application of automated gene synthesis techniques provides an opportunity for generating sequence variants of naturally occurring members of the B5 gene family. It will be appreciated, for example and as mentioned above, that polynucleotides coding for the B5 receptors herein described can be generated by substituting synonymous codons for those represented in the naturally occurring polynucleotide sequences herein identified. In addition, polynucleotides coding for synthetic variants of the B5 receptors herein described can be generated which incorporate one or more single amino acid substitutions, deletions or additions. Since it will for the most part be desirable to retain the natural ligand binding profile of the receptor for screening purposes, it is desirable to limit amino acid substitutions to the so-called conservative replacements in which amino acids of like charge are substituted, and to limit substitutions to those sites less critical for receptor activity. Alignment of the rat and human B5 protein sequences provided herein reveals amino acids that may be so modified without loss of receptor function and therefore, regions of B5 encoding nucleic acid at which can be varied without loss of function of the encoded B5 receptor. With reference to the rat B5 sequence of FIG. 1 and the numbering appearing thereon, amino acids 3, 11, 16, 20-22, 24-26, 29, 30, 39, 48, 51, 75, 97, 110, 114, 119, 120, 160, 191, 205, 214, 218, 222, 227, 237, 245, 251, 252, 253, 256, 257, 259, 260, 261, 263, 290, 292, 299, 302, 303, 308, 309, 315, 323, 349, 352, 353, 355-357, 360, 367, 368, 371, 375, 377, 379, 395, 397, 411-413, 415, 421, 423 and 431 may be modified without loss of function.

Alternatively, with appropriate template DNA in hand, the technique of PCR amplification may also be used to directly generate all or part of the final gene. In this case, primers are synthesized which will prime the PCR amplification of the final product, either in one piece, or in several pieces that may be ligated together. This may be via step-wise ligation of blunt-ended, amplified DNA fragments, or preferentially via step-wise ligation of fragments containing naturally occurring restriction endonuclease sites. In this application, it is possible to use either cDNA or genomic DNA as the template for the PCR amplification. In the former case, the cDNA template can be obtained from commercially available or self-constructed cDNA libraries of various human brain tissues, including hypothalamus and hind brain.

Once obtained, the receptor-encoding DNA is incorporated for expression into any suitable expression vector, and host cells are transfected therewith using conventional procedures, such as DNA-mediated transformation, electroporation, microinjection, or particle gun transformation. Expression vectors may be selected to provide transformed cell lines that express the receptor-encoding DNA either transiently or in a stable manner. For transient expression, host cells are typically transformed with an expression vector harbouring an origin of replication functional in a mammalian cell. For stable expression, such replication origins are unnecessary, but the vectors will typically harbour a gene coding for a product that confers on the transformants a survival advantage, to enable their selection. Genes coding for such selectable markers include the E. coli gpt gene which confers resistance to mycophenolic acid, the neo gene from transposon Tn5 which confers resistance to the antibiotic G418 and to neomycin, the dhfr sequence from murine cells or E. coli which changes the phenotype of DHFR− cells into DHFR+ cells, and the tk gene of herpes simplex virus, which makes TK− cells phenotypically TK+ cells. Both transient expression and stable expression can provide transformed cell lines, and membrane preparations derived therefrom, for use in ligand screening assays.

For use in screening assays, cells transiently expressing the receptor-encoding DNA can be stored frozen for later use, but because the rapid rate of plasmid replication will lead ultimately to cell death, usually in a few days, the transformed cells should be used as soon as possible. Such assays may be performed either with intact cells, or with membrane preparations derived from such cells. The membrane preparations typically provide a more convenient substrate for the ligand binding experiments, and are therefore preferred as binding substrates. To prepare membrane preparations for screening purposes, i.e., ligand binding experiments, frozen intact cells are homogenized while in cold binding buffer suspension and a membrane pellet is collected after centrifugation. The membranes may then be used as such, or after storage in lyophilized form, in the ligand binding assays. Alternatively, intact, fresh cells harvested about two days after transient transfection or after about the same period following fresh plating of stably transfected cells, can be used for ligand binding assays by the same methods as used for membrane preparations. When cells are used, the cells must be harvested by more gentle centrifugation so as not to damage them, and all washing must be done in a buffered medium, for example in phosphate-buffered saline, to avoid osmotic shock and rupture of the cells.

In an alternative to using cells that express receptor-encoding DNA, ligand characterization may also be performed using cells, for example Xenopus oocytes, that yield functional membrane-bound receptor following introduction of messenger RNA coding for the B5 receptor. In this case, the B5 receptor gene of the invention is typically subcloned into a plasmidic vector such that the introduced gene may be easily transcribed into RNA via an adjacent RNA transcription promoter supplied by the plasmidic vector, for example the T3 or T7 bacteriophage promoters. RNA is then transcribed from the inserted gene in vitro, and can then be injected into Xenopus oocytes. Following the injection of nL volumes of an RNA solution, the oocytes are left to incubate for up to several days, and are then tested in either intact or membrane preparations form for the ability to bind a particular ligand molecule supplied in a bathing solution.

The interaction of a candidate ligand with a selected B5 receptor of the invention is evaluated typically by determining receptor/ligand binding. In one embodiment, the interaction of ligands with a B5 receptor of the present invention can be determined by measuring a functional receptor/ligand interaction such as an electrophysiological interaction, by screening test ligands for their ability to modulate ion channel activity. The present invention thus further provides, as a ligand screening technique, a method of detecting interaction between a test ligand and a B5 receptor, which comprises the steps of incubating the test ligand with a B5 receptor-producing cell or with a membrane preparation derived therefrom, and then measuring ligand-induced electrical current across said cell or membrane using microelectrodes inserted into the cell or placed on either side of a cell-derived membrane preparation using the “patch-clamp” technique or a microphysiometer.

The interaction of a ligand with a B5 receptor can also be determined by assaying second messenger response associated with the B5 receptor activity to determine the ability of a given ligand to modulate B5 receptor activity. Furthermore, such second messenger response provides a means to differentiate antagonistic ligands from agonistic ligands. Such second messengers include, for example, cyclic AMP (cAMP) and intracellular calcium ion (Ca++). Thus, depending on the nature of the interaction, i.e. stimulatory or inhibitory, an increase or a decrease in intracellular cAMP or Ca++ can be measured to determine the extent of receptor/ligand interaction, using established assays. In a preferred embodiment, a B5 receptor-expressing cells in accordance with the present invention is subjected to adenylyl cyclase stimulant treatment, e.g. with forskolin, followed by incubation with a candidate ligand and a labelled substrate for adenylyl cyclase, e.g. [³²P]ATP, and then determining the extent of ligand-induced adenylyl cyclase activity, e.g. by determining the conversion of [³²P]ATP to [³²P]cAMP. Techniques such as those described in Salomon et al. in Anal. Biochem., 1974, 58:541 are useful to determine the conversion of ATP to cAMP.

In addition to using the receptor-encoding DNA to construct cell lines useful for ligand screening, expression of the DNA can, according to another aspect of the invention, be performed to produce fragments of the receptor in soluble form, for structure investigation, to raise antibodies and for other experimental uses. It will be appreciated that the production of such fragments may be accomplished in a variety of host cells. Mammalian cells such as CHO cells may be used for this purpose, the expression typically being driven by an expression promoter capable of high-level expression, for example the CMV (cytomegalovirus) promoter. Alternately, non-mammalian cells, such as insect Sf9 (Spodoptera frugiperda) cells may be used, with the expression typically being driven by expression promoters of the baculovirus, for example the strong, late polyhedrin protein promoter. Filamentous fungal expression systems may also be used to secrete large quantities of such domains of the B5 receptor. Aspergillus nidulans, for example, with the expression being driven by the alcA promoter, would constitute such an acceptable system. In addition to such expression hosts, it will be further appreciated that any prokaryotic or other eukaryotic expression system capable of expressing heterologous genes or gene fragments, whether intracellularly or extracellularly would be similarly acceptable.

For use particularly in detecting the presence and/or location of an B5 receptor, for example in brain tissue, the present invention also provides, in another of its aspects, labelled antibody to a human B5 receptor. To raise such antibodies, there may be used as immunogen either the intact, soluble receptor or an immunogenic fragment thereof, produced in a microbial or mammalian cell host as described above or by standard peptide synthesis techniques. Regions of the B5 receptor particularly suitable for use as immunogenic fragments include those corresponding in sequence to an extracellular region of the receptor, or a portion of the extracellular region, such as peptides consisting of residues 1-45, and peptides corresponding to the region between transmembrane domains TM-2 and TM-3, such as a peptide consisting of residues 103-117, between transmembrane domains TM-4 and TM-5, such as a peptide consisting of residues 180-214 and between transmembrane domains TM-6 and TM-7, such as a peptide consisting of residues 296-310. Peptides derived from intracellular loop domains are also appropriate for use in raising antibodies such as peptides corresponding to the region between transmembrane domains TM-1 and TM-2, such as residues 70-74, the region between transmembrane domains TM-3 and TM-4, such as residues 140-157, and the region between transmembrane domains TM-5 and TM-6, such as residues 238-272.

Peptides consisting of the C-terminal domain 335433, or fragments thereof may also be used for the raising of antibodies.

The raising of antibodies to the desired B5 receptor or immunogenic fragment can be achieved, for polyclonal antibody production, using immunization protocols of conventional design, and any of a variety of mammalian hosts, such as sheep, goats and rabbits. Alternatively, for monoclonal antibody production, immunocytes such as splenocytes can be recovered from the immunized animal and fused, using hybridoma technology, to myeloma cells. The fusion products are then screened by culturing in a selection medium, and cells producing antibody are recovered for continuous growth, and antibody recovery. Recovered antibody can then be coupled covalently to a detectable label, such as a radiolabel, enzyme label, luminescent label or the like, using linker technology established for this purpose.

In detectably labelled form, e.g. radiolabelled form or non-radiolabelled forms such as chemiluminescent forms, DNA or RNA coding for human and rat B5 receptors, and selected regions thereof, may also be used, in accordance with another aspect of the present invention, as hybridisation probes for example to identify sequence-related genes resident in the human or other mammalian genomes (or cDNA libraries) or to locate B5-encoding DNA in a specimen, such as brain tissue. This can be done using either the intact coding region, or a fragment thereof having radiolabelled nucleotides, e.g. ³²P, incorporated therein. To identify the B5-encoding DNA in a specimen, it is desirable to use either the full length cDNA coding therefor, or a fragment which is unique thereto: preferably, such fragments are at least 15 nucleotides long. Such unique regions can be identified by aligning the rat and human B5 nucleotide sequences provided herein with the nucleotide sequences of the most closely related known G protein coupled receptors. With reference to FIG. 1 and the nucleotide numbering appearing thereon, such nucleotide fragments include nucleotides 650-684; 702-726; 1155-1178;1190-1209;1264-1279;1368-1384 and 1391-1406. These sequences, and the intact gene itself, may also be used to clone B5 related human genes, particularly cDNA equivalents thereof, by standard hybridisation or PCR homology amplification techniques. Embodiments of the invention are described in the following specific examples which are not to be construed as limiting.

### EXAMPLE 1

Isolation of Nucleic Acid Encoding the Rat B5 Receptor

Two degenerate oligonucleotides P1 [5′-TTYGCNGTYWGCTGGHTSCC-3′]) and P2 (5′-TTIAGGMAISCGTARAWI ADDGGRTT-3′) (Y=C or T, W=A or T, S=C or G, M=A or C, R=A or G, D=A,G or T, H=A,C or T, N=A,C,G orT, I=inosine) were used as primers to amplify sequences from rat pancreatic mRNA using RT-PCR. Total RNA from rat pancreas was converted to single-stranded complementary DNA (cDNA) with random hexanucleotide primers using reverse transcriptase (Superscript II; Life Technologies, Inc. Catalog. No. 18053-017) according to the manufacturers recommendations. Subsequently, an aliquot of the single-stranded cDNA was amplified using PCR with primers P1 and P2 as follows: 30 seconds at 94° C. followed by 30 cycles of 94° C. for 30 seconds, 50° C. for 30 seconds and 72° C. for 1 minute. An aliquot of the PCR reaction was electrophoresed on a 1.5% agarose gel and the region of the gel corresponding to approximately 100-200 basepairs (bp) was excised and purified. The extracted DNA was reamplified with primers P1 and P2 uas follows: 30 seconds at 94° C. followed by 30 cycles of 94° C. for 30 seconds, 55° C. for 30 seconds and 72° C. for 1 minute. An aliquot of the PCR reaction was directly ligated into the vector pCR2.1 (nvitrogen catalog. No. 450046) and transformed into bacteria (Top10F° Catalog. No. C665-03). The resulting clone was sequenced by the dideoxy chain termination method on an Applied Biosystems Model 377 fluorescent dye DNA sequencer. This approach resulted in the discovery of a novel partial sequence. The full sequence, termed B5, of this partial which includes the entire open reading frame encoding for this putative novel GPCR receptor was obtained using two PCR-based techniques. Briefly, based on the sequence of clone B5, the following oligonucleotides were designed: P3, [5′-GGTGCTGCTGCTGCTCATCGACTAT-3′]; P4, [5′-TGGAAGAAGGCCAGCCAGTGTGCCAA-3′]; P5, [5′-TTGCAGCTCGCTCAGCTCCCCATA-3′]; and P6, [5′-TTGGCACACTGGCTGGCCTTCTTCCA -3′]. These oligonucleotides were used in an 5′ RACE (Innes et al. 1990: PCR protocols Academic Press Inc.) and Inverse PCR (PCR protocols, Supra) procedure to obtain sequences upstream and downstream of the sequence present in the B5 clone, respectively. The 5′ RACE technique was implemented to obtain upstream sequences from rat brain cDNA (Marathon-Ready™ cDNA, Clontech Laboratories Inc.; Cat No. 7470-1) using the Marathon™ cDNA Amplification Kit (Clontech Laboratories Inc.; Cat No. K1802-1) according to the manufacturers recommendations. Briefly, rat brain cDNA was amplified using primer P4 and the adaptor primer, AP1, (5′-CCATCCTAATACGACTCACTATAGGGC-3′; Clontech) under the following PCR conditions: 94° C. for 1 minute followed by 30 cycles at 94° C. for 30 seconds; 68° C. for 2.5 minutes and elongated at 68° C. for 7 minutes. An aliquot of this PCR reaction was used in a second PCR reaction with primer AP1 and the nested B5-specific primer P5. The PCR conditions were identical to those of the first PCR. An aliquot of this PCR reaction was electrophoresed on a 1.0% agarose gel. A faint band of 1.15 kb was visible by ethidium bromide staining. The band was gel purified and reamplified with AP1 and P5 under the following conditions: 1 minute at 94° C.; 5 cycles of 94° C. for 30 seconds; 72° C. for 3 minutes; 5 cycles of 94° C.; 70° C. for 3 minutes; 10 cycles of 94° C. for 30 seconds; 68° C. for 3 minutes. An aliquot of the PCR reaction was run on an 0.8% agarose gel and the 1.15 kb fragment was gel purified, ligated into pCR2.1 (nvitrogen) and transformed into Top10F′ bacterial cells. The resulting clones were sequenced as above. This 1.15 kb clone, called B5-5, overlapped the clone B5 and included sequences representing the entire 5′ end of this novel GPCR. B5-5 included the codon representing the initiating methionine as well as some 5′ UTR sequences.

The 3′ end of the novel GPCR was obtained using a Inverse PCR technique. Briefly, rat genomic DNA (Promega G313A) was restriction digested using one of four restriction endonucleases; EcoRI, BamHl, Hind III or Pst I (New England Biolabs & Pharmacia). The digested genomic DNA was purified and ligated at low concentration (2.7 ng/ul) to promote circularization of the DNA into monomeric circles (vectorettes). Following ligation, the DNAs were precipitated, concentrated and used as the template DNA for subsequent PCR reactions. In the initial PCR reaction P3 and P7 [5′-GATGCGCACGTACATCACTACCTA -3′] were used as primers. The PCR reaction was done as follows: 94° C. for 30 seconds, 61° C. for 30 seconds and 68° C. for 8 minutes for 30 cycles. An aliquot of these PCR reactions were used in a second PCR reaction using P7 and the nested primer P6. The reaction conditions were as follows: 94° C. for 1 minute; and 30 cycles of 94° C. for 30 seconds, 61° C. for 30 seconds and 68° C. for 3 minutes. Aliquots of the 4 PCR reactions were electrophoresed on a 1% agarose gel. A single intense band approximately 1.3 kb in size was visible by ethidium bromide staining in a lane correponding to the DNA which was initially digested with Pst I. This band was excised, purified, ligated into the vector pCR2.1 (Invitrogen) and transformed into bacteria (Top10F′). The resulting clones were sequenced as above. This 1.3 kb clone, called B5-3, overlapped the clone Beth-5 and included sequences representing the entire 3′ end of this novel GPCR including the stop codon. This sequence also included some 3′ UTR sequences. The sequence representing the full-length novel GCPR herein termed “B5” is shown in FIG. 1.

Reconstruction of a Full-Length Rat B5 Clone Using PCR

The DNA sequence encoding the novel GPCR was amplified using oligonucleotide primers corresponding to the 5′ and 3′ end of the cDNA. The 5′ oligonucleotide primer, termed PB5-5, has the sequence 5′-GGGGTTTAAGCTTGCCGCCACCATGGGTCCAATAGGTGCAGA GG-3′ and contains a EcoRI restriction site and a consensus Kozak translation initiation sequence followed by 24 nucleotides of the B5 sequence starting from the codon following the methionine start codon. The 3′ oligonucleotide primer, termed PB5-3, has the sequence 5′-GGGGAATTCATCCATACATTTTCACACCAC-3′ and contains 36 bases of the 3′ UTR of the B5 sequence with two mutations introduced to a Xbal restriction site. The two primers were used to amplify the full-length B5 from rat pancreas cDNA using Vent Polymerase (New England Biolabs) according to the manufacturers recommended procedure. The PCR reaction was done as follows: 7 minutes at 98° C. followed by 30 cycles of 94° C. for 1 minute, 60° C. for 1 minute and 72° C. for 2 minutes. An aliquot of the PCR reaction was restriction digested with the enzymes EcoRI and HindIII and electrophoresed on an 1% agarose gel. The PCR product was excised, purified, ligated into the EcoRI/Xbal sites of the mammalian expression vector pDI-neo (Promega) resulting in a ‘recombinant DNA construct”, named pCI-B5. Orientation of the cDNA was confirmed by restriction digestion analysis and sequencing.

### EXAMPLE 2

Isolation of Nucleic Acid Encoding the Human B5 Rreceptor

The human homologue of rat B5 was obtained by screening BAC filters (Genome Systems Inc., Cat No. BAC-5131) using the rat clone B5-5 as a probe according to the manufacturers recommendations except for the following modifications. The filters were prehybridized in 6× SSPE, 0.5% SDS, 0.1 mg/nL heparin, and 25% formamide for 3 h at 50° C. Next, hybridization was performed overnight at 50° C. in fresh prehybridization buffer with ³²P-labelled B5-5 cDNA (1×10⁶ cpm/mL. The filters were washed to a stringency of 0.1× SSPE/0.1% SDS at 50° C. for 20 minutes and exposed overnight onto Kodak X-OMAT film. Three clones corresponding to the following positions, 248-F9, 122-M4, and 165-K5 were identified and purchased form Genome Systems Inc. BAC DNA was isolated using the Very Low-Copy Plasmid Purification protocol (QIAgen; Cat. No. 12143). To confirm that the BAC clones contained the human homolog of rat B5, the degenerate primers P3 and P4 were used to amplify the TM6 -TM7 region of B5 from the BAC clones under the following conditions: 94° C. for 1 minute, followed by 30 cycles of 94° C. for 30 seconds; 68° C. for 30 seconds; and 72° C. for 1 minute. A 100 bp band was observed form all 3 BAC clones. An aliquot of the PCR reaction was ligated into the vector pCR2.1 (Invitrogen) and transformed into Top10F′ bacterial cells. The resulting clones were sequenced as above. Based on the sequences obtained from the BAC clones representing human B5, the following human-specific synthetic oligonucleotides were designed: P8 (5′-GGGGAAGGCGTAGACGGTGACCAGGTGCAG-3′), P9 (5′-CTGCACCTGGTCA CCGTCTACGCCTTCCCC-3′), and P10 (5′-GGGCAGCTCAGCGCGCCGCA GCTGCACCTG-3′). Using these primers the sequence of BAC clones B122 and B248 were determined. The partial sequence of human B5 is represented in FIG. 2.

### EXAMPLE 3

Production of Mammalian Cells Transfected Expressing B5

The plasmid pCI-B5,described above, containing the entire coding region of the B5 receptor under the transcriptional control of the human CMV promoter was transfected into COS-1 cells using the ‘in suspension’ DEAE/dextran transfection method (Brakenhoff et al. 1994, Anal. biochem. 218: 46-463). Briefly, COS-1 cells were trypsinised and plated 1-2 days prior to transfection in a 25-cm² culture flask. The next day the cells were trypsinised, counted, and 1×10⁶ cells resuspended in 0.5 ml RSC:RPMI 1640, supplemented with 100 μM chloroquine and 2% FCS. DNA (1 μg/μl) was added to 2 ml RSC and mixed with 2 ml RSC-DEAE: 800 μg/ml DEAE dextran in RSC. After 2 min incubation at room temperature the cells resuspended in RSC were added, and the suspension was incubated for 2 h in the tissue culture incubator under 5% CO₂ at 37° C. The cells were subsequently spun at 800 g for 5 min and resuspended in 10 ml DMEM. 10⁶ cells were added to 18 or 2 ml DMEM and seeded on culture dishes.

### EXAMPLE 4A

Chromosomal Localization

The procedure for FISH detection was performed to determine the chromosomal localisation of the B5 receptor.

(a) Slides Preparation

Lymphocytes isolated from human blood were cultured in a-minimal essential medium (MEM) supplemented with 10% fetal calf serum and phytohemagglutinin (PHA) at 37° C. for 68-72 hr. The lymphocyte cultures were treated with BrdU (0.18 mg/ml Sigma) to synchronize the cell population. The synchronized cells were washed three times with serum free medium to release the block and recultured at 37° C. for 6 hr in a MEM with thymidine (2.5 μg/ml: Sigma). Cells were harvested and slides were made by using standard procedures including hypotonic treatment, fix and air-dry.

(b) In situ Hybridization and FISH Detection

BAC probe was biotinylated with dATP using the BRL BioNick labelling kit (15° C., 2 hr) (Heng et al, High Resolution Mapping of Mammalian Genes by in situ Hybridization to Free Chromatin. Proc. NatI Aca Sci USA 89: 9509-9513, 1992).

The procedure for FISH detection was performed according to Heng et al., 1992 and Heng and Tsui 1993 (Modes of DAPI banding and simultaneous in situ hybrization. Chromosoma. 102: 325-332 (1993)). Briefly, slides were baked at 55° C. for 1 hr. After RNase treatment, the slides were denatured in 70% formamide in 2× SSC for 2 min. in 70° C. followed by dehydration with ethanol. Probes were denatured at 75° C. for 5 min. in a hybridization mix consisting of 50% formamide and 10% dextran sulphate and human cot I DNA. Probes were loaded on the denatured chromosomal slides after 15 min incubation at 37° C. to suppress the repetitive sequences. After overnight hybridisation, slides were washed and detected as well as amplified. FISH signals and the DAPI banding pattern was recorded separately by taking photographs, and the assignment of the FISH mapping data with chromosomal bands was achieved by superimposing FISH signals with DAPI banded chromosomes (Heng and Tsui, 1993).

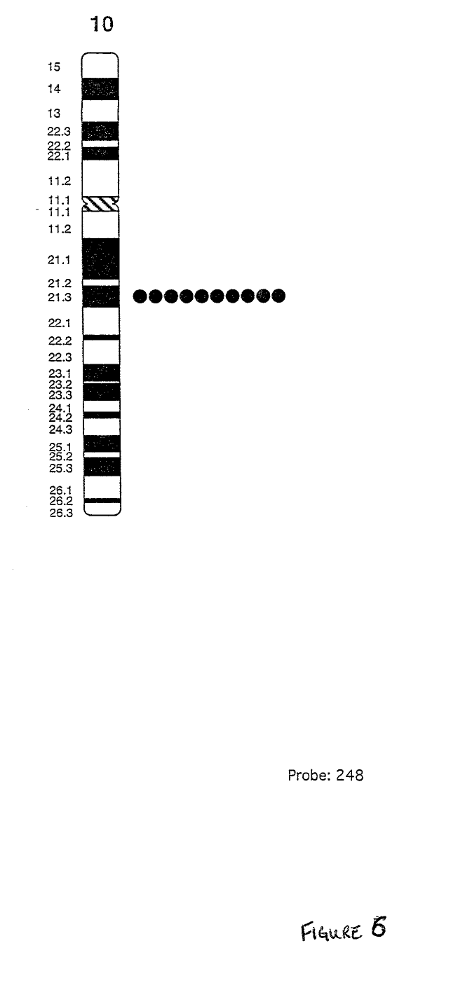

Two regions of one chromosome showed the FISH positive. Under the conditions used, the hybridisation efficiency was approximately 98% for this probe (among 100 checked mitotic figures, 98 of them showed signals on one pair of the chromosomes). Since the DAPI banding was used to identify the specific chromosome, the assignment between signal from probe and the long arm of chromosome 10 was obtained. The detailed position was further determined based on the summary from 10 photographs as set out in FIG. 6. There was no additional lock picked by FISH detection under the conditions used, therefore, probe 248 is located at human chromosome 10 region q21.

### EXAMPLE 4B

Testing of Prospective B5 Ligands of Binding to the B5 Receptor

Method A—Whole Cell Assay

COS-1 transfected as described hereinabove were grown on 2-well chamber slides (Nunc) (80% confluent), washed in phosphate-buffered saline and prehybridized in incubation buffer (Kreb's Phosphate Buffer (118 mM NaCl, 2.4 mM MgSO4, 4.7 mM Kcl, 0.59 mM KH2P04, 12.5 mM NaHCO3, and 1.7 mM CaCl2 )with 0.4% BSA and 0.05% Bacitracin) for 30 minutes at room temperature. For binding studies, the cells were incubated in the incubation buffer supplemented with the ¹²⁵I test ligand e.g. 100 pM ¹²⁵I-PYY or 100 pM ¹²⁵I-NPY (Amersham) at room temperature for 2 h. The slides were dipped sequentially 3-5 times in cold incubation minus BSA and Bacitracin for 5 seconds, rinsed in cold dH2O, and air dried before exposure to 3H-Hyperfilm (Amersham) 3 days.

Method B—Purified Membranes

Transfected COS-1 cells were grown on 150 mm petri dishes (80% confluent), washed in phosphate-buffered saline and homogenised in 5 volumes of ice-cold homogenisation buffer (25 mM HEPES, 2.5 mM CaCl2, 1 mM MgCl2, pH 7.4) using a Polytron homogenizer (set to 9500 rpm). Protein concentrations were measured using Coomassie protein assay reagent (Pierce) with BSA as a standard. Saturation experiments were performed with 50-100 ug of the whole cell lysate at room temperature for 2 hours using various concentration of ¹²⁵ labelled test ligand e.g. [¹²⁵I]-PYY (NEN) in a final volume of 200 ul of homogenisation buffer supplemented with 0.2% bacitracin. Non-specific binding was defined as the amount of radioactivity remaining bound to the cell homogenate after incubation in the presence of 1 mM unlabeled ligand,-in this case human NPY. The reaction was terminated by rapid filtration through Whatman GF/C filters, using a Tomtec (Orange, Conn.) cell harvester.

In competition studies, various concentrations of peptides: Human NPY, Porcine PYY13-36, Porcine NPY2-36, Porcine PYY3-36, Porcine [Leu31, Pro34]-NPY, Human PP (hPP), Rat PP (rPP) (Peninsula Laboratories Inc.) were included in the incubation mixture along with 0.25-0.5 nM [125I]-PYY. Both competition binding and saturation binding data were analysed by Prism program (GraphPad Software).

### EXAMPLE5

Antisense Analysis

Knowledge of the correct, complete cDNA sequence of B5 enables its use as a tool for antisense technology in the investigation of gene function. Oligonucleotides, cDNA or genomic fragments comprising the antisense strand of B5 are used either in vitro or in vivo to inhibit expression of the mRNA. Such technology is now well known in the art, and antisense molecules can be designed at various locations along the nucleotide sequences. By treatment of cells or whole test animals with such antisense sequences, the gene of interest is effectively turned off. Frequently, the function of the gene is ascertained by observing behavior at the intracellular, cellular, tissue or organismal level (e.g., lethality, loss of differentiated function, changes in morphology, etc.).

In addition to using sequences constructed to interrupt transcription of a particular open reading frame, modifications of gene expression is obtained by designing antisense sequences to intron regions, promoter/enhancer elements, or even to trans-acting regulatory genes. Similarly, inhibition is achieved using Hogeboom base-pairing methodology, also known as “triple helix” base pairing.

### EXAMPLE 6

Testing of Chimeric Seven Transmembrane G Protein Coupled Receptors

Functional chimeric seven transmembrane G protein coupled receptors (T7Gs) are constructed by combining the extracellular and/or transmembrane ligand-receptive sequences of a new isoform with the transmembrane and/or intracellular segments of a different T7G for test purposes. This concept was demonstrated by Kobilka et al (1988, Science 240:1310-1316) who created a series of chimeric α2-α2 adrenergic receptors (AR) by inserting progressively greater amounts of α2-AR transmembrane sequence into β2-AR. The binding activity of known agonists changed as the molecule shifted from having more α2 than β2 conformation, and intermediate constructs demonstrated mixed specificity. The specificity for binding antagonists, however, correlated with the source of the domain VII. The importance of T7G domain VII for ligand recognition was also found in chimeras utilizing two yeast a-factor receptors and is significant because the yeast receptors are classified as miscellaneous receptors. Thus, functional role of specific domains appears to be preserved throughout the T7G family regardless of category.

In parallel fashion, internal segments or cytoplasmic domains from a particular isoform are exchanged with the analogous domains of a known T7G and used to identify the structural determinants responsible for coupling the receptors to trimeric G-proteins (Dohlman et al (1991) Annu Rev Biochem 60:653-88). A chimeric receptor in which domains V, VI, and the intracellular connecting loop from β2-AR were substituted into a2-AR was shown to bind ligands with a2-AR specificity, but to stimulate adenylate cyclase in the manner of β2-AR. This demonstrates that for adrenergic-type receptors, G-protein recognition is present in domains V and VI and their connecting loop. The opposite situation was predicted and observed for a chimera in which the V→VI loop from α1-AR replaced the corresponding domain on β2-AR and the resulting receptor bound ligands with β2-AR specificity and activated G-protein-mediated phosphatidylinositol turnover in the α1-AR manner. Finally, chimeras constructed from muscarinic receptors also demonstrated that V→VI loop is the major determinant for specificity of G-protein activity.

Chimeric or modified T7Gs containing substitutions in the extracellular and transmembrane regions have shown that these portions of the receptor determine ligand binding specificity. For example, two Ser residues conserved in domain V of all adrenergic and D catecholamine T7G receptors are necessary for potent agonist activity. These serines are believed to form hydrogen bonds with the catechol moiety of the agonists within the T7G binding site. Similarly, an Asp residue present in domain III of all T7Gs which bind biogenic amines is believed to form an ion pair with the ligand amine group in the T7G binding site.

Functional, cloned T7Gs are expressed in heterologous expression systems and their biological activity assessed (e.g. Marullo et al (1988) Proc Natl Acad Sci 85:7551-55; King et al (1990) Science 250:121-23). One heterologous system introduces genes for a mammalian T7G and a mammalian G-protein into yeast cells. The T7G is shown to have appropriate ligand specificity and affinity and trigger appropriate biological activation, growth arrest and morphological changes, of the yeast cells.

An alternate procedure for testing chimeric receptors is based on the procedure utilizing the P₂ᵤ purinergic receptor (P₂ᵤ) as published by Erb et al (1993, Proc Natl Acad Sci 90:104411-53). Function is easily tested in cultured K562 human leukemia cells because these cells lack P₂ᵤ receptors. K562 cells are transfected with expression vectors containing either normal or chimeric P₂ᵤ and loaded with fura-a, fluorescent probe for Ca++. Activation of properly assembled and functional P2u receptors with extracellular UTP or ATP mobilizes intracellular Ca++ which reacts with fura-a and is measured spectrofluorometrically. As with the T7G receptors above, chimeric genes are created by combining sequences for extracellular receptive segments of any newly discovered T7G polypeptide with the nucleotides for the transmembrane and intracellular segments of the known P₂ᵤ molecule. Bathing the transfected K562 cells in microwells containing appropriate ligands triggers binding and fluorescent activity defining effectors of the T7G molecule. Once ligand and function are established, the P₂ᵤ system is useful for defining antagonists or inhibitors which block binding and prevent such fluorescent reactions.

### EXAMPLE 7

Diagnostic Test Using B5 Specific Antibodies

B5 antibodies are useful for investigating signal transduction and the diagnosis of infectious or hereditary conditions which are characterized by differences in the amount or distribution of B5 or downstream products of an active signaling cascade.

Diagnostic tests for B5 include methods utilizing antibody and a label to detect B5 in human body fluids, membranes, cells, tissues or extracts of such. The polypeptides and antibodies of the present invention are used with or without modification. Frequently, the polypeptides and antibodies are labeled by joining them, either covalently or noncovalently, with a substance which provides for a detectable signal. A wide variety of labels and conjugation techniques are known and have been reported extensively in both the scientific and patent literature. Suitable labels include radionuclides, enzymes, substrates, cofactors, inhibitors, fluorescent agents, chemiluminescent agents, chromogenic agents, magnetic particles and the like. Patents teaching the use of such labels include U.S. Pat. Nos. 3,817,837; 3,850,752; 3,939,350; 3,996,345; 4,277,437; 4,275,149; and 4,366,241. Also, recombinant immunoglobulins may be produced as shown in U.S. Pat. No. 4,816,567, incorporated herein by reference.

A variety of protocols for measuring soluble or membrane-bound Beth 5, using either polyclonal or monoclonal antibodies specific for the protein, are known in the art. Examples include enzyme-linked imnunosorbent assay (ELISA), radioimmunoassay (RIA) and fluorescent activated cell sorting (FACS). A two-site monoclonal-based immunoassay utilizing monoclonal antibodies reactive to two non-interfering epitopes on B5 is preferred, but a competitive binding assay may be employed. These assays are described, among other places, in Maddox, DE et al (1983, J Exp. Med. 158:1211f).

### EXAMPLE 8

Purification of Native B5 Using Specific Antibodies

Native or recombinant B5 is purified by immunoaffinity chromatography using antibodies specific for B5. In general, an immunoaffinity column is constructed by covalently coupling the anti-TRH antibody to an activated chromatographic resin.

Polyclonal immunoglobulins are prepared from immune sera either by precipitation with ammonium sulfate or by purification on immobilized Protein A (Pharmacia LKB Biotechnology, Piscataway N.J.). Likewise, monoclonal antibodies are prepared from mouse ascites fluid by ammonium sulfate precipitation or chromatography on immobilized Protein A. Partially purified immunoglobulin is covalently attached to a chmmatographic resin such as CnBr-activated Sepharose (Pharmacia LKB Biotechnology). The antibody is coupled to the resin, the resin is blocked, and the derivative resin is washed according to the manufacturer's instructions.

Such immunoaffinity columns are utilized in the purification of B5 by preparing a fraction from cells containing B5 in a soluble form. This preparation is derived by solubilization of whole cells or of a subcellular fraction obtained via differential centrifugation (with or without addition of detergent) or by other methods well known in the art. Alternatively, soluble B5 is secreted in useful quantity into the medium in which the cells are grown.

A soluble B5-containing preparation is passed over the immunoaffinity column, and the column is washed under conditions that allow the preferential absorbance of B5 (e.g., high ionic strength buffers in the presence of detergent). Then, the column is eluted under conditions that disruptantibody/protein binding (e.g., a buffer of pH 2-3 or a high concentration of a chaotrope such as urea or thiocyanate ion), and B5 is collected.

### EXAMPLE 10

Drug Screening

This invention is particularly useful for screening therapeutic compounds by using B5 or binding fragments thereof in any of a variety of drug screening techniques. As B5 is a G protein coupled receptor any of the methods commonly used in the art may potentially used to identify B5 ligands. For example, the activity of a G protein coupled receptor such as B5 can be measured using any of a variety of appropriate functional assays in which activation of the receptor results in an observable change in the level of some second messenger system, such as adenylate cyclase, guanylyl cyclase, calcium mobilization, or inositol phospholipid hydrolysis. One such approach, measures the effect of ligand binding on the activation of intracellular second messenger pathways, using a reporter gene. Typically, the reporter gene will have a promoter which is sensitive to the level of that second messenger controlling expression of an easily detectable gene product, for example, CAT or luciferase. Alternatively, the cell is loaded with a reporter substance, e.g., FURA whereby changes in the intracellular concentration of calcium indicate modulation of the receptor as a result of ligand binding. Thus, the present invention provides methods of screening for drugs or any other agents which affect signal transduction.

Alternatively, the polypeptide or fragment employed in such a test is either free in solution, affixed to a solid support, borne on a cell surface or located intracellularly. One method of drug screening utilizes eukaryotic or prokaryotic host cells which are stabley transformed with recombinant nucleic acids expressing the polypeptide or fragment. Drug candidates are screened against such transformed cells in competitive binding assays. Such cells, either in viable or fixed form, are used for standard binding assays. One measures, for example, the formation of complexes between B5 and the agent being tested. Alternatively, one examines the diminution in complex formation between B5 and a ligand caused by the agent being tested.

This invention also contemplates the use of competitive drug screening assays in which neutralizing antibodies capable of binding B5 specifically compete with a test compound for binding to B5 polypeptides or fragments thereof. In this manner, the antibodies are used to detect the presence of any peptide which shares one or more antigenic determinants with B5.

### EXAMPLE 12

Use and Administration of Antibodies, Inhibitors, or Antagonists

Antibodies, inhibitors, or antagonists of B5 (or other treatments to limit signal transduction, LST) provide different effects when administered therapeutically. LSTs are formulated in a nontoxic, inert, pharmaceutically acceptable aqueous carrier medium preferably at a pH of about 5 to 8, more preferably 6 to 8, although pH may vary according to the characteristics of the antibody, inhibitor, or antagonist being formulated and the condition to be treated. Characteristics of LSTs include solubility of the molecule, half-life and antigenicity/immunogenicity. These and other characteristics aid in defining an effective carrier.

LSTs are delivered by known routes of administration including but not limited to topical creams and gels; transmucosal spray and aerosol; transdermal patch and bandage; injectable, intravenous and lavage formulations; and orally administered liquids and pills particularly formulated to resist stomach acid and enzymes. The particular formulation, exact dosage, and route of administration is determined by the attending physician and varies according to each specific situation.

Such determinations are made by considering multiple variables such as the condition to be treated, the LST to be administered, and the pharmacokinetic profile of a particular LST. Additional factors which are taken into account include severity of the disease state, patient's age, weight, gender and diet, time and frequency of LST administration, possible combination with other drugs, reaction sensitivities, and tolerance/response to therapy. Long acting LST formulations might be administered every 3 to 4 days, every week, or once every two weeks depending on half-life and clearance rate of the particular LST.

Normal dosage amounts vary from 0.1 to 100,000 micrograms, up to a total dose of about 1 g, depending upon the route of administration. Guidance as to particular dosages and methods of delivery is provided in the literature; see U.S. Pat. Nos. 4,657,760; 5,206,344; or 5,225,212. Those skilled in the art employ different formulations for different LSTs. Administration to cells such as nerve cells necessitates delivery in a manner different from that to other cells such as vascular endothelial cells.

It is contemplated that abnormal signal transduction, trauma, or diseases which trigger B5 activity are treatable with LSTs. These conditions or diseases are specifically diagnosed by the tests discussed above, and such testing should be performed in suspected cases of viral, bacterial or fungal infections: allergic responses; mechanical injury associated with trauma; hereditary diseases; lymphoma or carcinoma; or other conditions which activate the genes of lymphoid or neuronal tissues.

### EXAMPLE 13

Production of Transgenic Animals

Animal model systems which elucidate the physiological and behavioral roles of the B5 receptor are produced by creating transgenic animals in which the activity of the B5 receptor is either increased or decreased, or the amino acid sequence of the expressed B5 receptor is altered, by a variety of techniques. Examples of these techniques include, but are not limited to: 1) Insertion of normal or mutant versions of DNA encoding a B5 receptor, by microinjection, electroporation, retroviral transfection or other means well known to those skilled in the art, into appropriate fertilized embryos in order to produce a transgenic animal or 2) Homologous recombination of mutant or normal, human or animal versions of these genes with the native gene locus in transgenic animals to alter the regulation of expression or the structure of these B5 receptor sequences. The technique of homologous recombination is well known in the art. It replaces the native gene with the inserted gene and so is useful for producing an animal that cannot express native B5 receptors but does express, for example, an inserted mutant B5 receptor, which has replaced the native B5 receptor in the animal's genome by recombination, resulting in underexpression of the transporter. Microinjection adds genes to the genome, but does not remove them, and so is useful for producing an animal which expresses its own and added B5 receptors, resulting in overexpression of the B5 receptors.

One means available for producing a transgenic animal, with a mouse as an example, is as follows: Female mice are mated, and the resulting fertilized eggs are dissected out of their oviducts. The eggs are stored in an appropriate medium such as M2 medium. DNA or cDNA encoding a B5 purified from a vector by methods well known in the art. Inducible promoters may be fused with the coding region of the DNA to provide an experimental means to regulate expression of the transgene. Alternatively or in addition, tissue specific regulatory elements may be fused with the coding region to permit tissue-specific expression of the trans-gene. The DNA, in an appropriately buffered solution, is put into a microinjection needle (which may be made from capillary tubing using a piper puller) and the egg to be injected is put in a depression slide. The needle is inserted into the pronucleus of the egg, and the DNA solution is injected. The injected egg is then transferred into the oviduct of a pseudopregnant mouse (a mouse stimulated by the appropriate hormones to maintain pregnancy but which is not actually pregnant), where it proceeds to the uterus, implants, and develops to term. As noted above, microinjection is not the only methods for inserting DNA into the egg cell, and is used here only for exemplary purposes.

All publications and patents mentioned in the above specification are herein incorporated by reference.

Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the above-described modes for carrying out the invention which are obvious to those skilled in the field of molecular biology or related fields are intended to be within the scope of the following claims.

We claim:

1. An isolated polynucleotide encoding a B5 receptor the polynucleotide selected from the group consisting of:
a) a polynucleotide encoding a polypeptide having the deduced amino acid seqence of FIG. 1 or a fragment, analog or derivative of said polypeptide; and
b) a polynucleotide capable of hybridising to and which is at least 70% identical the polynucleotide of FIG. 1.

2. An isolated polynucleotide according to claim 1, wherein the polynucleotide is the polynucleotide of FIG. 1.

3. An isolated polynucleotide encoding a polypeptide having the deduced amino acid sequence of FIG. 2 or a fragment, analog or derivative of said polypeptide.

4. An isolated polynucleotide according to claim 3, wherein said polynucleotide is the polynucleotide of FIG. 2.

5. An isolated polynucleotide comprising a region that encodes a variant of the B5 receptor, said variant sharing at least 84% amino acid identity with the B5 receptor.

6. A recombinant DNA construct having incorporated therein a polynucleotide as defined in any one of claims 1 to 5.

7. A cell that has been engineered genetically to produce a B5-binding human receptor, said cell having incorporated expressibly therein a recombinant construct as defined in claim 6.

8. A cell as defined in claim 7, which is a mammalian cell.

9. A B5-binding membrane preparation derived from a cell as defined in claim 8.

10. A method of assaying a test ligand for binding with a B5 receptor, which comprises the steps of incubating the test ligand under appropriate conditions with a B5 receptor-producing cell as defined in claim 7, or with membrane preparation derived therefrom, and then determining whether binding between the B5 receptor and the test ligand has occurred.

11. A method of assaying a test ligand for interaction with a B5 receptor, which comprises the steps of incubating the test ligand under appropriate conditions with a B5 receptor-producing cell as defined in claim 7, or with membrane preparation derived therefrom, and then determining the extent of interaction between the human B5 receptor and the test ligand by measuring a functional receptor response.

12. A method as defined in claim 11, wherein the functional receptor response is second messenger response.

13. A method as defined in claim 12, wherein said second messenger is selected from intracellular cAMP and intracellular calcium ion.

14. A B5 receptor, in a form essentially free from other proteins of human origin.

15. A ligand-binding fragment of a B5.

18. An antibody which binds a mammalian B5 receptor.

19. An immunogenic fragment of a human B5.

20. An oligonucleotide which comprises at least about 17 nucleic acids and which selectively hybridizes with a polynucleotide defined in claim 1 or complement thereof.


SEQUENCE LISTING (20 sequences)

SEQ ID NO: 1 - DNA, 1532 bp, Rattus sp.
gcgagtgacgggtgaagcaggaacgagggtaacccacccagaccccagacccttcctgggccccagtctacccgcttgaaggtgcccgcctcctttggagagtgtcccggagcagacagtatggaggcggagccctcccagcctcccaacggcagctggcccctgggtcagaacgggagtgatgtggagaccagcatagcaaccagcctcaccttctcctcctactgccaacactcctctccggtggcagccatgttcatcgcggcctacgtgctcatcttcctcctctgcatagtgggcaacaccctggtctacttcattgtgctcaagaaccggcacatgcgcactgtcaccaacatgtttatcctcaacctggccgtcagcgacctgccggtgggcatcttctgcatgcccacaacccttgtggacaaccttatcactggttggccttttgacaacgccacatgcaagatgagcggcttggtgcagggcatgtccgtgtctgcatcggttttcacactggtggccatcgctgtggaaaggttccgctgcatcgtgcaccctttccgcgagaagctgacccttcggaaggcgctgttcaccatcgcggtgatctgggctctggcgctgctcatcatgtgtccctcggcggtcactctgacagtcacccgagaggagcatcacttcatgctggatgctcgtaaccgctcctacccgctctactcgtgctggggggcctggcccgagaagggcatgcgcaaggtctacaccgcggtgctcttcgcgcacatctacctggtgccgctggcgctcatcgtagtgatgtacgtgcgcatcgcgcgcaagctatgccaggcccccggtcctgcgcgcgacacggaggaggcggtggccgagggtggccgcacttcgcgccgtagggcccgcgtggtgcacatgctggccatggtggcgctcttcttcacgttgtcctggctgccactctgggtgctgctgctgctcatcgactatggggagctgagcgagctgcaactgcacctgctgtcggtctacgccttccccttggcacactggctggccttcttccacagcagcgccaaccccatcatctacggctacttcaacgagaacttccgccgcggcttccaggctgccttccgtgcacagctctgctggcctccctgggccgcccacaagcaagcctactcggagcgacccaaccgcctcctgcgcaggcgggtggtggtggacgtgcaacccagcgactccggcctgccatcagagtctggccccagcagcggggtcccagggcctggccggctgccactgcgaaatgggcgtgtggcccatcaggatggcccgggggaagggccaggctgcaaccacatgcccctcaccatcccggcctggaacatttgaggtggtccagagaagggagggccagtagtcctgcggccctgacccttaactaagatgcccacgcacaatagcagtattagaagaaggtgccaagatgcctccttgataaaaaa

SEQ ID NO: 2 - protein, 432 aa, Rattus sp.
MEAEPSQPPNGSWPLGQNGSDVETSIATSLTFSSYCQHSSPVAAMFIAAYVLIFLLCIVGNTLVYFIVLKNRHMRTVTNMFILNLAVSDLPVGIFCMPTTLVDNLITGWPFDNATCKMSGLVQGMSVSASVFTLVAIAVERFRCIVHPFREKLTLRKALFTIAVIWALALLIMCPSAVTLTVTREEHHFMLDARNRSYPLYSCWGAWPEKGMRKVYTAVLFAHIYLVPLALIVVMYVRIARKLCQAPGPARDTEEAVAEGGRTSRRRARVVHMLAMVALFFTLSWLPLWVLLLLIDYGELSELQLHLLSVYAFPLAHWLAFFHSSANPIIYGYFNENFRRGFQAAFRAQLCWPPWAAHKQAYSERPNRLLRRRVVVDVQPSDSGLPSESGPSSGVPGPGRLPLRNGRVAHQDGPGEGPGCNHMPLTIPAWNI

SEQ ID NO: 3 - DNA, 1320 bp, Homo sapiens
ggggagccctcccagcctcccaacagcagttggcccctaagtcagaatgggactaacactgaggccaccccggctacaaacctcaccttctcctcctactatcagcacacctcccctgtggcggccatgttcattgtggcctatgcgctcatcttcctgctctgcatggtgggcaacaccctggtctgtttcatcgtgctcaagaaccggcacatgcatactgtcaccaacatgttcatcctcaacctggctgtcagtgacctgctggtgggcatcttctgcatgcccaccacccttgtggacaacctcatcactggttggccttttgacaacgccacatgcaagatgagcggcttggtgcagggcatgtccgtgtctgcatcggttttcacactggtggccatcgctgtggaaaggttccgctgcatcgtgcaccctttccgcgagaagctgaccctgcggaaggcgctcgtcaccatcgccgtcatctgggccctggcgctgctcatcatgtgtccctcggccgtcacgctgaccgtcacccgtgaggaacaccacttcatggtggacgcccgcaaccgctcctacccgctctactcctgctgggaggcctggcccgaaaagggcatgcgcagggtctacaccactgtgctcttctcgcacatctacctggcgccgctggcgctcatcgtggtcatgtacgcccgcatcgcgcgcaagctctgcaaggccccgggcccggcccccgggggcgaggaggctgcggacccgcgagcatcgcggcgcagagcgcgcgtggtgcacatgctggtcatggtggcgctgttcttcacgctgtcctggctgccgctctgggcgctgctgctgctcatcgactacgggcagctcagcgcgccgcagctgcacctggtcaccgtctacgccttccccttcgcgcactggctggccttcttcaacagcagcgccaaccccatcatctacggctacttcaacgagaacttccgccgcggcttccaggccgccttccgcgcccgcctctgcccgcgcccgtcggggagccacaaggaggcctactccgagcggcccggcgggcttctgcacaggcgggtcttcgtggtggtgcggcccagcgactccgggctgccctctgagtcgggccctagcagtggggcccccaggcccggccgcctcccgctgcggaatgggcgggtggctcaccacggcttgcccagggaagggcctggctgctcccacctgcccctcaccattccagcctgggatatctgagggggtccagggagggcgggacgctgcctccag

SEQ ID NO: 4 - protein, 428 aa, Homo sapiens
GEPSQPPNSSWPLSQNGTNTEATPATNLTFSSYYQHTSPVAAMFIVAYALIFLLCMVGNTLVCFIVLKNRHMHTVTNMFILNLAVSDLLVGIFCMPTTLVDNLITGWPFDNATCKMSGLVQGMSVSASVFTLVAIAVERFRCIVHPFREKLTLRKALVTIAVIWALALLIMCPSAVTLTVTREEHHFMVDARNRSYPLYSCWEAWPEKGMRRVYTTVLFSHIYLAPLALIVVMYARIARKLCKAPGPAPGGEEAADPRASRRRARVVHMLVMVALFFTLSWLPLWALLLLIDYGQLSAPQLHLVTVYAFPFAHWLAFFNSSANPIIYGYFNENFRRGFQAAFRARLCPRPSGSHKEAYSERPGGLLHRRVFVVVRPSDSGLPSESGPSSGAPRPGRLPLRNGRVAHHGLPREGPGCSHLPLTIPAWDI

SEQ ID NO: 5 - protein, 381 aa, Homo sapiens
MGPIGAEADENQTVEEMKVEQYGPQTTPRGELVPDPEPELIDSTKLIEVQVVLILAYCSIILLGVIGNSLVIHVVIKFKSMRTVTNFFIANLAVADLLVNTLCLPFTLTYTLMGEWKMGPVLCHLVPYAQGLAVQVSTITLTVIALDRHRCIVYHLESKISKRISFLIIGLAWGISALLASPLAIFREYSLIEIIPDFEIVACTEKWPGEEKSIYGTVYSLSSLLILYVLPLGIISFSYTRIWSKLKNHVSPGAANDHYHQRRQKTTKMLVCVVVVFAVSWLPLHAFQLAVDIDSQVLDLKEYKLIFTVFHIIAMCSTFANPLLYGWMNSNYRKAFLSAFRCEQRLDAIHSEVSVTFKAKKNLEVRKNSGPNDSFTEATNV

SEQ ID NO: 6 - DNA, 20 bp, Artificial Sequence
  Description of Artificial Sequenceprimer
ttygcngtywgctgghtscc

SEQ ID NO: 7 - DNA, 26 bp, Artificial Sequence
  Description of Artificial Sequenceprimer
ttaaggmaascgtarawaaddggrtt

SEQ ID NO: 8 - DNA, 25 bp, Artificial Sequence
  Description of Artificial Sequenceprimer
ggtgctgctgctgctcatcgactat

SEQ ID NO: 9 - DNA, 26 bp, Artificial Sequence
  Description of Artificial Sequenceprimer
tggaagaaggccagccagtgtgccaa

SEQ ID NO: 10 - DNA, 24 bp, Artificial Sequence
  Description of Artificial Sequenceprimer
ttgcagctcgctcagctccccata

SEQ ID NO: 11 - DNA, 26 bp, Artificial Sequence
  Description of Artificial Sequenceprimer
ttggcacactggctggccttcttcca

SEQ ID NO: 12 - DNA, 27 bp, Artificial Sequence
  Description of Artificial Sequenceprimer
ccatcctaatacgactcactatagggc

SEQ ID NO: 13 - DNA, 24 bp, Artificial Sequence
  Description of Artificial Sequenceprimer
gatgcgcacgtacatcactaccta

SEQ ID NO: 14 - DNA, 44 bp, Artificial Sequence
  Description of Artificial Sequenceprimer
ggggtttaagcttgccgccaccatgggtccaataggtgcagagg

SEQ ID NO: 15 - DNA, 30 bp, Artificial Sequence
  Description of Artificial Sequenceprimer
ggggaattcatccatacattttcacaccac

SEQ ID NO: 16 - DNA, 30 bp, Artificial Sequence
  Description of Artificial Sequenceprimer
ggggaaggcgtagacggtgaccaggtgcag

SEQ ID NO: 17 - DNA, 30 bp, Artificial Sequence
  Description of Artificial Sequenceprimer
ctgcacctggtcaccgtctacgccttcccc

SEQ ID NO: 18 - DNA, 30 bp, Artificial Sequence
  Description of Artificial Sequenceprimer
gggcagctcagcgcgccgcagctgcacctg

SEQ ID NO: 19 - protein, 426 aa, Rattus sp.
MEAEPSQPPNGSWPLGQNGSDVETSIATSLTFSSYCQHSSPVAAMFIAAYVLIFLLCIVGNTLVYFIVLKNRHMRTVTNMFILNLAVSDLPVGIFCMPTTLVDNLITGWPFDNATCKMSGLVQGMSVSASVFTLVAIAVERFRCIVHPFREKLTLRKALFTIAVIWALALLIMCPSAVTLTVTREEHHFMLDARNRSYPLYSCWGAWPEKGMRKVYTAVLFAHIYLVPLALIVVMYVRIARKLCQAPGPARDTEEAVAEGGRTSRRRARVVHMLAMVALFFTLSWLPLWVLLLLIDYGELSELQLHLLSVYAFPLAHWLAFFHSSANPIIYGYFNENFRRGFQAAFRAQLCWPPWAAHKQAYSERPNRLLRRRVVVDVQPSDSGLPSESGPSSGVPGPGRLPLRNGRVAHQDGPGEGPGCNHMPLT

SEQ ID NO: 20 - protein, 381 aa, Homo sapiens; identical to SEQ ID NO: 5